SEQUENCE LISTING (10 sequences)

SEQ ID NO: 1 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 2 - protein, 258 aa, Papio hamadryas
MWFLVLCLALSLGGTGAAPPIQSRIVGGWECSQPWQAALYHFSTFQCGGILVHPQWVLTAAHCIGDNYQLWLGRHNLFDDEDTAQFVHVSESFPHPCFNMSLLKNHTRQADEDYSHDLMLLRLTQPAEITDAVQVVELPTQEPEVGSTCLASGWGSIEPENFSYPDDLQCVDLKILPNDKCAKAHTQKVTEFMLCAGHLEGGKDTCVGDSGGPLTCDGVLQGVTSWGYIPCGSPNKPAVFVRVLSYVKWIEDTIAENS

SEQ ID NO: 3 - protein, 257 aa, Macaca fascicularis
MWFLVLCLALSLGGTGRAPPIQSRIVGGWECSQPWQAALYHFSTFQCGGILVHPQWVLTAAHCISDNYQLWLGRHNLFDDEDTAQFVHVSESFPHPGFNMSLLKNHTRQADDYSHDLMLLRLTQPAEITDAVQVVELPTQEPEVGSTCLASGWGSIEPENFSFPDDLQCVDLEILPNDECAKAHTQKVTEFMLCAGHLEGGKDTCVGDSGGPLTCDGVLQGVTSWGYIPCGSPNKPAVFVKVLSYVKWIEDTIAENS

SEQ ID NO: 4 - protein, 261 aa, Saguinus oedipus
MWFLVLCLALSLGGTGAVPPIQSRIVGGWDCKQHSQPWQAALYHYSTFQCGGVLVHPQWVLTAAHCISDHYQLWLGRHDLFENEDTAQFVFVSKSFPHPDFNMSLLKNHTRLPGEDYSHDLMLLQLKQPVQITDAVKVVELPTEGIEVGSTCLASGWGSIKPEKFSFPDILQCVDLKILPNDECDKAHAQKVTEFMLCAGPLKDGQDTCVGDSGGPLTCDGVLQGIISWGYIPCGSPNKPSVFVRVLSYVKWIKDTIADNS

SEQ ID NO: 5 - protein, 261 aa, Canine lupus
MWFLVLCLALSLAGTGAAPPVQSRIIGGWDCTKNSQPWQAALYHYSKFQCGGVLVHPEWVVTAAHCINDNYQLWLGRYNLFEHEDTAQFVQVRESFPHPEFNLSLLKNHTRLPEEDYSHDIMLLRLAEPAQITDAVRVLDLPTQEPQVGSTCYASGWGSIEPDKFIYPDDLQCVDLELLSNDICANAHSQKVTEFMLCAGHLEGGKDTCVGDSGGPLICDGVLQGITSWGHVPCGSPNMPAVYTKVISHLEWIKETMTANP

SEQ ID NO: 6 - protein, 261 aa, Ovus aries
MWFPVLCLALSLAGTGAVPPVQSRIVGGQECEKHSQPWQVAIYHFSTFQCGGVLVAPQWVLTAAHCKSENYQVWLGRHNLFEDEDTAQFAGVSEDFPNPGFNLSLLENHTRQPGEDYSHDLMLLRLQEPVQLTQDVQVLGLPTKEPQLGTTCYASGWGSVKPDEFSYPDDLQCVDLTLLPNEKCATAHPQEVTDCMLCAGHLEGGKDTCVGDSGGPLICEGMLQGITSWGHIPCGTPNKPSVYTKVIVYLDWINKTMTDNP

SEQ ID NO: 7 - protein, 261 aa, Oryctolagus cuniculus
MWLPVLCLALSLGGTGAAPPLQSRIIGGWVCGKNSQPWQAALYHYSNFQCGGVLVHPQWVLTAAHCFSDNYQLWLGRHNLFEDEAEAQFIQVSGSFPHPRFNLSLLENQTRGPGEDYSHDLMLLKLARPVQLTNAVRVLELPTQEPQVGTSCLASGWGSITPIKFTYPDELQCVDLSILANSECDKAHAQMVTECMLCAGHLEGGRDTCVGDSGGPLVCNNELQGITSWGHVPCGSPNKPAVFTKVLSYVEWIRNTIANNP

SEQ ID NO: 8 - protein, 261 aa, Bos taurus
MWFPVLCLALSLAGTGAVFPIQSRIVGGQECEKHSQPWQVAIYHFSTFQCGGVLVAPQWVLTAAHCKSDNYQVWLGRHNLFEDEDTAQFAGVSEDFPNPGFNLSLLENHTRHPGEDYSHDLMLLRLQEPVQLTQNVQVLGLPTKEPQLGTTCYASGWGSVKPDEFSYPDDLQCVDLTLLPNEKCATAHPQEVTEWMLCAGHLEGGKDTCVGDSGGPLICEGMLQGITSWGHIPCGTPNKPSVYTKVILYLDWINKTMTDNP

SEQ ID NO: 9 - protein, 261 aa, Equus caballus
MWLPVLCLALSLVGTGAAPPIQSRIIGGWECKNHSKPWQAAVYHYSSFQCGGVLVDPQWVLTAAHCKGDYYQIWLGRHNLFEDEDTAQFFLVAKSFPHPDFNMSLLENNNRLPGEDYSHDLMLLQVEQPDQITVAVQVLALPTQEPVLGSTCYASGWGSIEPDKFTYPDELRCVDLTLLSNDVCDNAHSQNVTEYMLCAGHLEGGKDTCVGDSGGPLICDGVFQGVTSWGHIPCGRPNKPAVYTKLIPHVQWIQDTIAANP

SEQ ID NO: 10 - protein, 263 aa, Sus scrofa
MWSLVMRLALSLAGTGAAPPIQSRIIGGRECEKDSHPWQVAIYHYSSFQCGGVLVDPKWVLTAAHCKNDNYQVWLGRHNLFENEVTAQFFGVTADFPHPGFNLSLLKNHTKADGKDYSHDLMLLRLQSPAKITDAVKVLELPTQEPELGSTCQASGWGSIEPGPDDFEFPDEIQCVELTLLQNTFCADAHPDKVTESMLCAGYLPGGKDTCMGDSGGPLICNGMWQGITSWGHTPCGSANKPSIYTKLIFYLDWINDTITENP

(19) United States
(12) Patent Application Publication
(10) Pub. No.: US 2012/0070425 A1
(43) Pub. Date: Mar. 22, 2012

(54) TISSUE KALLIKREIN FOR THE TREATMENT OF PANCREATIC Beta-CELL DYSFUNCTION AND FOR Beta-CELL PROLIFERATION

(75) Inventors: Mark WILLIAMS, Winnipeg (CA); Kevin Richardson, Winnipeg (CA)

(73) Assignee: DIAMEDICA, INC., Winnipeg (CA)

(21) Appl. No.: 13/241,882

(22) Filed: Sep. 23, 2011

Related U.S. Application Data
(63) Continuation-in-part of application No. PCT/CA2010/000413, filed on Mar. 25, 2010.
(60) Provisional application No. 61/163,173, filed on Mar. 25, 2009.

(51) Int. Cl. A61K 38/48; A61P 3/08; A61P 3/10

(52) U.S. Cl. 424/94.64

(57) ABSTRACT

The invention relates to methods of administering kallikrein, a variant, or active fragment thereof to stimulate proliferation of islet cells generally and β-cells specifically. The invention also includes compositions to stimulate proliferation in vivo and in vitro.

### CROSS REFERENCE TO RELATED APPLICATIONS

This application is a continuation-in-part of International Application No. PCT/CA2010/000413, filed March 25, 2010, which claims the benefit under 35 U.S.C. §119(e) of U.S. provisional application no. 61/163,173, filed Mar. 25, 2009, the entire disclosures of which are hereby incorporated by reference.

### FIELD OF THE INVENTION

The present invention relates to methods of the treatment of pancreatic β-cell dysfunction and treating pancreatic diseases and conditions associated therewith by the modulation β-cells.

### BACKGROUND OF THE INVENTION

Both type I and type II diabetes mellitus are of great concern today. Approximately 24 million people in the United States are affected by the disease (Mueller, Phys Ther, 2008, 88(11):1250-3) and the incidence is on the rise around the globe. While type I diabetes can only be treated by insulin injections, type II diabetes may be treated through diet and exercise in some cases. Diet and exercise can even ward off type II diabetes development; however, the increasing sedentary lifestyle in many regions of the world is resulting in obesity at epidemic proportions. For those who require it, treatment can be expensive and inconvenient, and may produce several undesirable side effects with the currently available drugs.

Diabetes can be diagnosed by various means. For instance, a fasting plasma glucose (FPG) test measures blood glucose in a person who has not eaten anything for at least 8 hours. The FPG test is the preferred test for diagnosing diabetes because of its convenience and low cost. An oral glucose tolerance test (OGTT) measures blood glucose after a person fasts at least 8 hours and 2 hours after the person drinks a glucose-containing beverage. Both FPG and OGTT can also be used to assess prediabetes.

A random plasma glucose test, also called a casual plasma glucose test, measures blood glucose without regard to when the person being tested last ate. This test, along with an assessment of symptoms, is used to diagnose diabetes. Test results indicating that a person has diabetes should be confirmed with a second test on a different day.

Gestational diabetes can also be diagnosed based on plasma glucose values measured during an OGTT, preferably by using 100 grams of glucose in liquid for the test. Blood glucose levels are checked four times during the test. If blood glucose levels are above normal at least twice during the test, the woman is diagnosed as having gestational diabetes. Above-normal results for the OGTT for gestational diabetes are indicated by 95 mg/dL at fasting, 180 mg/dL at 1 hour, 155 mg/dL at 2 hours, and 140 mg/dL at 3 hours (when using 100 g glucose).

A random, or casual, blood glucose level of 200 mg/dL or higher, plus symptoms of increased urination, increased thirst, and/or unexplained weight loss, can mean a person has diabetes. Other symptoms can include fatigue, blurred vision, increased hunger, and sores that do not heal. A physician can check the person's blood glucose level on another day using the FPG test or the OGTT to confirm the diagnosis.

Glycated hemoglobin (hemoglobin A1c, HbA1c, A1C, or Hb1c; sometimes also HbA1c) is a form of hemoglobin which is measured primarily to identify the average plasma glucose concentration over prolonged periods of time. The 2010 American Diabetes Association Standards of Medical Care in Diabetes added the HbA1C test A1c≧48 mmol/mol (≧6.5%) as another criterion for the diagnosis for diabetes.

β-cells, found in the pancreas, are responsible for the production and release of insulin into the blood stream. They represent the majority of the endocrine cells and form the core of the islets. The pancreatic β-cells secrete insulin in response to increasing glucose levels. Insulin aids in the entry of glucose into the muscle and fat cells. (Ellingsgaard et al, PNAS, 2008, 105(35): 13162-7). In individuals with type I diabetes, the β-cells are attacked by an autoimmune response. The β-cells that remain are insufficient to produce enough insulin to remove the glucose from the blood. They show increased levels of β-cell destruction. For those with type II diabetes, the muscle and liver cells are no longer able to respond to normal blood insulin levels. Therefore they also end up with high blood glucose levels. This can result in β-cell death and loss of β-cell function as compared to healthy individuals. Long standing cases of type I diabetes show a ˜99% deficit in β-cell mass while cases of type II diabetes show a ˜65% deficit in β-cell mass within the pancreas (Meier, Diabetologia, 2008, 51:703-13). Modulation of the levels of healthy β-cells, in particular, increasing the levels or activity of such cells, may therefore serve as an effective therapy to reverse and possibly prevent diabetes.

Modulation of β-cell levels has been attempted through the use of stem cells and organ transplant. These methods are seen to have some drawbacks (Meier, Diabetologia, 2008, 51:703-13). Issues regarding stem cell supply and ethics may limit the ability of this therapy. Transplant presents the risks associated with any organ transplant, be it rejection, infection and/or subsequent mortality.

A therapy is therefore desired which can stimulate β-cell production thereby increasing the β-cell mass and improving blood glucose. This strategy will effectively serve to suppress glucagon secretion (Ellingsgaard et al, PNAS, 2008, 105(35): 13162-7) and restore insulin production and secretion to normal levels, resulting in further suppression of glucagon secretion and hepatic glucose production, leading to an improvement in overall peripheral insulin action (Meier, Diabetologia, 2008, 51:703-13).

Until the recent work of the present inventors, no one has contemplated the use of tissue kallikrein (KLK1) for the treatment of pancreatic islet β-cell dysfunction and treating diseases and conditions associated therewith by the modulation β-cell mass. KLK1 is a serine protease which cleaves low-molecular-weight kininogen resulting in the release of kallidin (lysl-bradykinin) KLK1 may be formulated to produce a product which can be delivered to modulate β-cell mass without any of the issues associated with the alternate proposed therapies (stem cell usage and pancreatic organ transplant).

### SUMMARY OF THE INVENTION

The present invention includes methods of the treatment of pancreatic islet β-cell dysfunction and treating diseases and conditions associated therewith by the modulation β-cell mass comprising administering a therapeutically effective dose of KLK1, variants of KLK1, or active fragments thereof.

Embodiments of the present invention include a method of treating a human subject to stimulate β cell production comprising administering about 0.01 to about 100 IU per day of tissue kallikrein-1 (KLK1), a variant of active fragment thereof to the subject. The KLK1 can be administered via subcutaneous, intramuscular, or intravenous administration. In embodiments, the subject is newly diagnosed with diabetes, and/or the subject is treated to improve blood glucose levels. In embodiments, the amount of KLK1, a variant of active fragment thereof, is about 0.01 to about 10 IU per day.

In other embodiments, a method of stimulating β cell proliferation comprising contacting β cells with an effective amount of tissue kallikrein-1 (KLK1), a variant or active fragment thereof.

In one aspect of the present invention, the KLK1 can be an isolated natural form, an isolated synthetic form, or an isolated recombinant form.

In another aspect of the present invention, the isolated KLK1 can be human KLK1 (SEQ ID NO: 1 or SEQ ID NO:2).

In an embodiment of the present invention, preferably, the isolated KLK1 can be pig KLK1 (SEQ ID NO: 11).

In addition, the present invention further provides pharmaceutical compositions and method of treating a disease associated with reduced pancreatic islet β-cell functioning and/or reduced pancreatic islet β-cell mass.

In a preferred embodiment the disease or condition associated with reduced pancreatic islet β-cell functioning and/or reduced pancreatic islet β-cell mass is type I or type II diabetes.

In a further aspect of the invention, modulation of β-cell mass can be an increase in the β-cell mass as compared to the diseased state.

In a further aspect of the invention modulation of β-cell mass can be β-cell regeneration. In the present invention stimulation of β-cell replication refers to the restoration of normal β-cell function by increasing the number of functional β-cells or by fixing impaired β-cell by restoring normal function.

In yet a further aspect of the present invention modulation of β-cell mass can be an increase in β-cell replication.

Another aspect of the present invention includes a method as herein described further comprising the use of an additional therapeutic method useful in the modulation of β-cell mass or treating diseases or conditions associated therewith. An additional therapeutic method includes, but is not limited to, stem cell transplant and pancreatic organ transplant.

Another aspect of the present invention includes a composition formulated for oral administration comprising about 1 to about 1000 IU of KLK1, or a variant or an active fragment thereof, optionally further comprising a pharmaceutically acceptable excipient, and optionally further comprising an additional therapeutic compound as described above.

### BRIEF DESCRIPTION OF THE FIGURES

FIG. 1 is a graph of pancreatic β cell area (insulin positive) as a percentage of total pancreas area in the various KLK1 dosage groups in STZ-treated rats on day 28. Data are presented as mean ±SD.**p<0.01

FIG. 2 is a graph of pancreatic β cell mass (mg) in the various KLK1 dosage groups in STZ-treated rats on day 28. Data are presented as mean±SD.**p<0.01

FIG. 3 is a graph of the number of pancreatic islets (from two 4× objective fields) in the various KLK1 dosage groups in STZ-treated rats on day 28. Data are presented as mean±SD.

FIG. 4 is a graph of pancreatic β cell proliferation in islets as assessed by BrdU and insulin double-positive cells in the various KLK1 dosage groups in STZ-treated rats on day 28. Data are presented as mean±SD.#p<0.01 compared to the STZ+PBS group.

FIG. 5 is a graph of the number of insulin-positive duct cells in the various KLK1 dosage groups in STZ-treated rats on day 28. Data are presented as mean±SD.

FIG. 6 shows photomicrographs (40× magnification) of pancreas histology in the various KLK1 dosage groups. Pancreatic islets are stained bright red via insulin immunohistochemistry.

FIG. 7 is a graph of fasting blood glucose levels (mg/dL) on Days 5, 8, 14, 21, and 27 in the various KLK1 dosage groups as indicated. Data are presented as mean±SEM. Blood samples were obtained from a tail nick in conscious rats.

FIG. 8 shows statistical significance of the fasting blood glucose levels data in FIG. 7 for the no STZ group compared to the other groups (top panel), and the lower panel shows significance data for the STZ+PBS group compared to the other groups (lower panel). *p<0.05; **p<0.01; ***p<0.001

FIG. 9 is a graph showing insulin content (ng/g) in the tail portion of the pancreas on Day 28 in the various KLK1 dosage groups. The bars from left side of the figure (closest to y axis) are as follows: 1)No STZ+PBS 2) STZ+PBS, 3) STZ, KLK1 0.2 U, 4) STZ, KLK1 1.0 U, and 5) STZ, KLK1 5.0 U. Data are presented as mean±SEM.

FIG. 10 are graphs for the oral glucose tolerance test (OGTT) on Days 14 (FIG. 10A), 21 (FIG. 10B), and 27 (FIG. 10C) in the various KLK1 dosage groups as indicated. Time points were 0, 15, 30, 60, 90, and 120 minutes after injection of a glucose load. Data are presented as mean±SEM. Rats were fasted for overnight for the Day 14 time point and 6 hours for the Day 21 and 27 time points.

FIG. 11 is a graph for peak glucose levels in the OGTT for the various KLK1 dosage groups. Data are presented as mean±SEM. The middle panel shows statistical significance data for the no STZ group compared to the other groups, and the lower panel shows significance data for the STZ+PBS group compared to the other groups. *p<0.05; **p<0.01; ***p<0.001

FIG. 12A is a graph for positive peak area under the curve (AUC) for the OGTTs performed on Days 14, 21, and 27 in the various KLK1 dosage groups. Data are presented as mean±SEM. FIG. 12B shows statistical significance data for the no STZ group compared to the other groups (upper panel), and for the STZ+PBS group compared to the other groups (lower panel). *p<0.05; **p<0.01; ***p<0.001

FIG. 13A is a graph for total AUC for the OGTTs performed on Days 14, 21, and 27 in the various KLK1 dosage groups. Data are presented as mean±SEM. FIG. 13B shows statistical significance data for the no STZ group compared to the other groups (upper panel), and for the STZ+PBS group compared to the other groups (lower panel). *p<0.05; **p<0.01; ***p<0.001 FIG. 14 is a graph for post-prandial insulin levels (ng/mL) after a glucose load in the various KLK1 dosage groups at Day 27. Data points were 0, 15, 30, 60, 90, and 120 minutes after injection of a glucose load. Data are presented as mean±SEM.

FIG. 15 is a graph for positive peak AUC for OGTTs performed on Day 27 for blood insulin content in the various KLK1 dosage groups. The bars from left side of the figure (closest to y axis) to right are as follows: 1) No STZ+PBS 2) STZ+PBS 3) STZ, KLK1 0.2 U, 4) STZ, KLK1 1.0 U, and 5) STZ, KLK1 5.0 U. Data are presented as mean±SEM. There were no significant differences between the groups.

FIG. 16 is a graph showing urinary glucose excretion (mg/day) in the various KLK1 dosage groups on Day 25. The bars from left side of the figure (closest to y axis) to right are as follows: 1) No STZ+PBS; 2) STZ+PBS; 3) STZ, KLK1 0.2 U; 4) STZ, KLK1 1.0 U; and 5) STZ, KLK1 5.0 U. Data are presented as mean±SEM.

FIG. 17 is a graph showing urinary protein excretion in the various KLK1 dosage groups on Day 25. The bars from left side of the figure (closest to y axis) to right are as follows: 1) No STZ+PBS; 2) STZ+PBS; 3) STZ, KLK1 0.2 U; 4) STZ, KLK1 1.0 U; and 5) STZ, KLK1 5.0 U. Data are presented as mean±SEM.

FIG. 18 is a graph showing urinary albumin excretion in the various KLK1 dosage groups on Day 25. The bars from left side of the figure (closest to y axis) to right are as follows: 1) No STZ+PBS; 2) STZ+PBS; 3) STZ, KLK1 0.2 U; 4) STZ, KLK1 1.0 U; and 5) STZ, KLK1 5.0 U. Data are presented as mean±SEM.

FIG. 19 is a graph showing the ratio of total kidney weight to body weight in the various KLK1 dosage groups on Day 25. The bars from left side of the figure (closest to y axis) to right are as follows: 1) No STZ+PBS; 2) STZ+PBS; 3) STZ, KLK1 0.2 U; 4) STZ, KLK1 1.0 U; and 5) STZ, KLK1 5.0 U. Data are presented as mean±SEM.

### DETAILED DESCRIPTION

The practice of the present invention will employ, unless otherwise indicated, conventional methods of microbiology, molecular biology and recombinant DNA techniques within the skill of the art. Such techniques are explained fully in the literature. See, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition).

The various compositions and methods of the invention are described in detail below. Although particular compositions and methods are exemplified herein, it is understood that any of a number of alternative compositions and methods are applicable and suitable for use in practicing the invention.

### Definitions

Unless otherwise indicated, all terms used herein have the same meaning as they would to one skilled in the art, and the practice of the present invention will employ conventional techniques of microbiology and recombinant DNA technology, which are within the knowledge of those of skill in the art.

The term “amino acid” is used in its broadest sense and is meant to include the naturally occurring L α-amino acids or residues. The commonly used one and three letter abbreviations for naturally occurring amino acids are used herein (Lehninger, 1975, Biochemistry, 2d ed., pp. 71-92, Worth Publishers, New York). The term also includes all D-amino acids as well as chemically modified amino acids such as amino acid analogs, naturally occurring amino acids that are not usually incorporated into proteins such as norleucine, and chemically synthesized compounds having properties known in the art to be characteristic of an amino acid. For example, analogs or mimetics of phenylalanine or proline, which allow the same conformational restriction of the peptide compounds as natural Phe or Pro are included within the definition of amino acid. Such analogs and mimetics are referred to herein as “functional equivalents” of an amino acid. Other examples of amino acids are listed by Roberts and Vellaccio, In: The Peptides: Analysis, Synthesis, Biology, Gross and Meiehofer, Eds., Vol. 5 p 341, Academic Press, Inc, N.Y. 1983, which is incorporated herein by reference.

The term “protein” has an amino acid sequence that is longer than a peptide. A “peptide” contains 2 to about 50 amino acid residues. The term “polypeptide” includes proteins and peptides. Examples of proteins include, but are not limited to, antibodies, enzymes, lectins and receptors; lipoproteins and lipopolypeptides; and glycoproteins.

The term “recombinant” as used herein refers to nucleic acids, vectors, polypeptides, or proteins that have been generated using DNA recombination (cloning) methods and are distinguishable from native or wild-type nucleic acids, vectors, polypeptides, or proteins. A “variant” of a polypeptide or polynucleotide refers to a polypeptide that contains an amino acid sequence or a polynucleotide that contains a nucleotide sequence that differs from a reference sequence. The reference sequence can be a full-length native polypeptide or polynucleotide sequence or any other fragment of a full-length polypeptide or polynucleotide sequence. An embodiment of a reference sequence is SEQ ID NO:1. A polypeptide or polynucleotide variant generally has at least about 80% 85%, 90%, 91%, 92%, 93%, 94%, 95%, 96%, 97%, 98%, 98.5%, 99%, or 99.5% amino acid or nucleotide sequence identity with a reference sequence. It should be noted, however, that to qualify as a “variant” such polypeptide or polynucleotide sequence must differ from a reference sequence in at least one amino acid residue or nucleotide. Embodiments include variants of KLK1. A variant of KLK1 includes a polypeptide comprising at least one of a deletion, insertion, or substitution, or combinations thereof, of an amino acid relative to a reference polypeptide sequence (i.e., SEQ ID NO:1). For example, a variant of KLK1 could include a polypeptide with one, two, or three deletions, insertions, or substitutions, or combinations thereof.

As used herein, the term “conserved amino acid substitutions” refers to the substitution of one amino acid for another at a given location in the protein, where the substitution can be made without substantial loss of the relevant function. In making such changes, substitutions of like amino acid residues can be made on the basis of relative similarity of side-chain substituents, for example, their size, charge, hydrophobicity, hydrophilicity, and the like, and such substitutions may be assayed for their effect on the function of the protein by routine testing.

A “wild type” or “reference” sequence or the sequence of a “wild type” or “reference” protein/polypeptide may be the reference sequence from which variant polypeptides are derived through the introduction of mutations. In general, a “wild type” sequence for a given polypeptide is the sequence that is most common in nature. Similarly, a “wild type” gene sequence is the sequence for that gene which is most commonly found in nature. Mutations may be introduced into a “wild type” gene (and thus the protein it encodes) either through natural processes or through man induced means. The products of such processes are “variant” or “mutant” forms of the original “wild type” protein or gene.

“Percent (%) amino acid sequence identity” with respect to a polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, Calif.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y,

where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

“Percent (%) nucleic acid sequence identity” is defined as the percentage of nucleotides in a candidate sequence that are identical with the nucleotides in a reference polypeptide-encoding nucleic acid sequence, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

The term “active fragment” refers to smaller portions of a polypeptide that retain an activity of the full-length or mature polypeptide.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

The term “therapeutically effective amount” refers to an amount of a composition of this invention effective to “alleviate” or “treat” a disease or disorder in a subject or mammal. Generally, alleviation or treatment of a disease or disorder involves the lessening of one or more symptoms or medical problems associated with the disease or disorder. In some embodiments, the therapeutically effective amount is an amount that increases pancreatic β cell mass, increases pancreatic β cell area, results in replication of β cells in islets, or results in an increase the number of insulin positive duct cells. In other embodiments, the amount may improve or lower fasting blood glucose levels, or increase pancreatic insulin content. In other embodiments, the amount may improve blood glucose levels during an oral glucose tolerance test (OGTT), or lower peak glucose levels during an OGTT, or lower the total blood glucose during an OGTT. In another embodiment, the amount may increase blood insulin levels or increase the amount of blood insulin content after a meal or during an OGTT.

The terms “treating” or “treatment” is an approach for obtaining beneficial or desired clinical results. Herein, beneficial or desired clinical results include, but are not limited to, alleviation or inhibition of symptoms, diminishment of extent of disease, stabilization (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” is an intervention performed with the intention of preventing the development or altering the pathology of a disorder. “Treating” or “treatment” does not refer to “prophylactic” or “preventative” measures. In some embodiments, the treatment may result in increased pancreatic β cell mass, increased pancreatic β cell area, increased replication of β cells in islets, or an increased number of insulin positive duct cells. In other embodiments, treatment may result in an improvement or lowering of fasting blood glucose levels or increased pancreatic insulin content. In other embodiments, treatment may result in an improvement in blood glucose levels during an oral glucose tolerance test (OGTT), lower peak glucose levels during an OGTT, or lowered total blood glucose during an OGTT. In another embodiment, treatment may result in increased blood insulin levels or increased amount of blood insulin content after a meal or during an OGTT. In another embodiment, treatment may result in decreased levels of HbA1C from elevated levels, defined as HbA1C levels≧48 mmol/mol (≧6.5%).

The terms “preventing”, “preventative”, or “prophylactic” refer to an administration of a therapeutic embodiment wherein the object is to prevent the targeted pathologic condition or disorder. Thus, “prevention” includes preventing a disease or symptoms from occurring or delaying the onset of a disease or symptoms.

The term “significant” or “significantly”, as used herein, refers to statistical significance. Statistical significance is P<0.05, unless a correction is applied to correct for a confounding influence (e.g., Bonferroni's correction).

The term “physiologically acceptable” as used herein refers to molecular entities and compositions that are physiologically tolerable and do not typically produce toxicity or an allergic or similar untoward reaction, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated.

The term “about” or “approximately” means within a statistically meaningful range of a value. Such a range can be within an order of magnitude, preferably within 50%, more preferably within 20%, more preferably still within 10%, and even more preferably within 5% of a given value or range. The allowable variation encompassed by the term “about” or “approximately” depends on the particular system under study, and can be readily appreciated by one of ordinary skill in the art.

The term “newly diagnosed” refers to being diagnosed with diabetes for the first time by a health care professional. Newly diagnosed subjects include those that have had an initial diagnosis of diabetes within one year, 6 months, 3 months, 2 months, 1 month, 3 weeks, 2 weeks, or 1 week or less from the initiation of treatment.

The term “international unit” refers to an internationally accepted amount of a biologically active substance required to produce a specific response. All international units are officially defined by the International Conference for Unification of Formulae. The term “international unit” can be abbreviated as “IU” or “U” in English, UI in French and Italian, and IE in German.

### Tissue Kallikrein-1

“Tissue kallikrein-1” or “KLK1” is a serine protease that is primarily noted for its role in controlling hypertension through its cleavage of kininogen into lysyl-bradykinin (kallidin) (Yousef et al., Endocrine Rev. 2001; 22: 184-204). As there are a large number of enzymes in the KLK family, it is thought that KLK1 appears to be a ubiquitous or multiple target acting enzyme, in addition to its recognized role in hypertension regulation. As used herein, the term “tissue kallikrein” is synonymous with the following terms: callicrein, glumorin, padreatin, padutin, kallidinogenase, bradykininogenase, panceatic kallikrein, onokrein P, dilminal D, depot-Padutin, urokallikrein, or urinary kallikrein.

Exemplary sequences of human tissue kallikrein polypeptides include:
NP 002248 GI:4504875 Homo sapiens

1-18 signal peptide

19-24 propeptide

25-262 mature protein

gi|4504875|ref|NP_002248.1| kallikrein-1 prepro-
protein [Homo sapiens]
(SEQ ID NO: 1)
MWFLVLCLALSLGGTGAAPPIQSRIVGGWECEQHSQPWQAALYHFSTFQC
GGILVHRQWVLTAAHCISDNYQLWLGRHNLFDDENTAQFVHVSESFPHPG
FNMSLLENHTRQADEDYSHDLMLLRLTEPADTITDAVKVVELPTEEPEVG
STCLASGWGSIEPENFSFPDDLQCVDLKILPNDECKKAHVQKVTDFMLCV
GHLEGGKDTCVGDSGGPLMCDGVLQGVTSWGYVPCGTPNKPSVAVRVLSY
VKWIEDTIAENS

Hamadryas baboon tissue kallikrein (kidney/pancreas/salivary gland kallikrein) (SEQ ID NO: 2) which has 90% sequence identity to human KLK 1 (SEQ ID NO: 1)
Q28773.1 GI:3024066 Papio hamadryas

1-18 signal peptide

19-24 propeptide

25-258 mature protein

gi|3024066|sp|Q28773.1|KLK1_PAPHA
(SEQ ID NO: 2)
MWFLVLCLALSLGGTGAAPPIQSRIVGGWECSQPWQAALYHFSTFQCGGI
LVHPQWVLTAAHCIGDNYQLWLGRHNLFDDEDTAQFVHVSESFPHPCFNM
SLLKNHTRQADEDYSHDLMLLRLTQPAEITDAVQVVELPTQEPEVGSTCL
ASGWGSIEPENFSYPDDLQCVDLKILPNDKCAKAHTQKVTEFMLCAGHLE
GGKDTCVGDSGGPLTCDGVLQGVTSWGYIPCGSPNKPAVFVRVLSYVKWI
EDTIAENS

Crab eating macaque tissue kallikrein (kidney/pancreas/salivary gland kallikrein) (SEQ ID NO: 3) which has 90% sequence identity to human KLK1 (SEQ ID NO: 1):
Q07276.1 GI:585360 Macaca fascicularis

1-18 signal peptide

19-24 propeptide

25-257 mature protein

gi|585360|sp|Q07276.1|KLK1_MACFA
(SEQ ID NO: 3)
MWFLVLCLALSLGGTGRAPPIQSRIVGGWECSQPWQAALYHFSTFQCGGI
LVHPQWVLTAAHCISDNYQLWLGRHNLFDDEDTAQFVHVSESFPHPGFNM
SLLKNHTRQADDYSHDLMLLRLTQPAEITDAVQVVELPTQEPEVGSTCLA
SGWGSIEPENFSFPDDLQCVDLEILPNDECAKAHTQKVTEFMLCAGHLEG
GKDTCVGDSGGPLTCDGVLQGVTSWGYIPCGSPNKPAVFVKVLSYVKWIE
DTIAENS

Cotton top tamarin tissue kallikrein (kidney/pancreas/salivary gland kallikrein) (SEQ ID NO : 4) which has 82% sequence identity to human KLK1 (SEQ ID NO: 1)

gi|75074638|sp|Q9N1Q1|Q9N1Q1_Saguinus Oedipus
Tissue kallikrein
(SEQ ID NO: 4)
MWFLVLCLALSLGGTGAVPPIQSRIVGGWDCKQHSQPWQAALYHYSTFQC
GGVLVHPQWVLTAAHCISDHYQLWLGRHDLFENEDTAQFVFVSKSFPHPD
FNMSLLKNHTRLPGEDYSHDLMLLQLKQPVQITDAVKVVELPTEGIEVGS
TCLASGWGSIKPEKFSFPDILQCVDLKILPNDECDKAHAQKVTEFMLCAG
PLKDGQDTCVGDSGGPLTCDGVLQGIISWGYIPCGSPNKPSVFVRVLSYV
KWIKDTIADNS

Dog tissue kallikrein (kidney/pancreas/salivary gland kallikrein) (SEQ ID NO. 5) which has 74% sequence identity to human KLK 1 (SEQ ID NO: 1):
Q29474 GI:75069518 Canis lupus familiaris

1-24 signal peptide

25-261 mature protein

gi|75069518|sp|Q29474|Q29474_CANFA Kallikrein
precursor
(SEQ ID NO: 5)
MWFLVLCLALSLAGTGAAPPVQSRIIGGWDCTKNSQPWQAALYHYSKFQC
GGVLVHPEWVVTAAHCINDNYQLWLGRYNLFEHEDTAQFVQVRESFPHPE
FNLSLLKNHTRLPEEDYSHDIMLLRLAEPAQITDAVRVLDLPTQEPQVGS
TCYASGWGSIEPDKFIYPDDLQCVDLELLSNDICANAHSQKVTEFMLCAG
HLEGGKDTCVGDSGGPLICDGVLQGITSWGHVPCGSPNMPAVYTKVISHL
EWIKETMTANP

Sheep tissue kallikrein-1 (SEQ ID NO: 7) which has 72% sequence identity to human KLK1 (SEQ ID NO: 1)
NP 001087256.1 GI:148232148 Ovis aries

1-24 signal peptide

25-261 mature protein

gi|148232148|ref|NP_001087256.1| kallikrein-1
[Ovis aries]
(SEQ ID NO: 6)
MWFPVLCLALSLAGTGAVPPVQSRIVGGQECEKHSQPWQVAIYHFSTFQC
GGVLVAPQWVLTAAHCKSENYQVWLGRHNLFEDEDTAQFAGVSEDFPNPG
FNLSLLENHTRQPGEDYSHDLMLLRLQEPVQLTQDVQVLGLPTKEPQLGT
TCYASGWGSVKPDEFSYPDDLQCVDLTLLPNEKCATAHPQEVTDCMLCAG
HLEGGKDTCVGDSGGPLICEGMLQGITSWGHIPCGTPNKPSVYTKVIVYL
DWINKTMTDNP

Rabbit tissue kallikrein-1 (SEQ ID NO: 7) which has 73% sequence identity to human KLK1 (SEQ ID NO:1)
NP 001093441.1 GI:153792063 Oryctolagus cuniculus

1-24 signal peptide

25-261 mature protein

gi|153792063|ref|NP_001093441.1| kallikrein 1
[Oryctolagus cuniculus]
(SEQ ID NO: 7)
MWLPVLCLALSLGGTGAAPPLQSRIIGGWVCGKNSQPWQAALYHYSNFQC
GGVLVHPQWVLTAAHCFSDNYQLWLGRHNLFEDEAEAQFIQVSGSFPHPR
FNLSLLENQTRGPGEDYSHDLMLLKLARPVQLTNAVRVLELPTQEPQVGT
SCLASGWGSITPIKFTYPDELQCVDLSILANSECDKAHAQMVTECMLCAG
HLEGGRDTCVGDSGGPLVCNNELQGITSWGHVPCGSPNKPAVFTKVLSYV
EWIRNTIANNP

Bovine glandular kallikrein-1 precursor (SEQ ID NO: 9) which has 72% sequence identity to human KLK1 (SEQ ID NO: 1)
Q6H320 GI:75071532 Bos taurus

1-17 signal peptide

18-24 propeptide

25-261 mature protein

gi|75071532|sp|Q6H3201|Q6H320_BOVIN Glandular
kallikrein precursor
(SEQ ID NO: 8)
MWFPVLCLALSLAGTGAVFPIQSRIVGGQECEKHSQPWQVAIYHFSTFQC
GGVLVAPQWVLTAAHCKSDNYQVWLGRHNLFEDEDTAQFAGVSEDFPNPG
FNLSLLENHTRHPGEDYSHDLMLLRLQEPVQLTQNVQVLGLPTKEPQLGT
TCYASGWGSVKPDEFSYPDDLQCVDLTLLPNEKCATAHPQEVTEWMLCAG
HLEGGKDTCVGDSGGPLICEGMLQGITSWGHIPCGTPNKPSVYTKVILYL
DWINKTMTDNP

Horse glandular kalikrein precursor (KLKE1) (SEQ ID NO. 9) which has 70% sequence identity to human KLK1 (SEQ 1D NO: 10)
Q6H322 GI:75071533 Equus caballus

1-17 signal peptide

18-23 propeptide

24-261 mature protein

gi|75071533|sp|Q6H322|Q6H322_HORSE Glandular
kallikrein precursor
(SEQ ID NO: 9)
MWLPVLCLALSLVGTGAAPPIQSRIIGGWECKNHSKPWQAAVYHYSSFQC
GGVLVDPQWVLTAAHCKGDYYQIWLGRHNLFEDEDTAQFFLVAKSFPHPD
FNMSLLENNNRLPGEDYSHDLMLLQVEQPDQITVAVQVLALPTQEPVLGS
TCYASGWGSIEPDKFTYPDELRCVDLTLLSNDVCDNAHSQNVTEYMLCAG
HLEGGKDTCVGDSGGPLICDGVFQGVTSWGHIPCGRPNKPAVYTKLIPHV
QWIQDTIAANP

Pig glandular kallikrein precursor (SEQ ID NO : 10) which has 67% sequence identity to human KLK1 (SEQ ID NO: 1):
NP_{—}001001911 GI:50054435 Sus scrofa

1-17 signal peptide

18-24 propeptide

25-263 mature protein

gi|50054435|ref|NP_001001911.1| kallikrein 1 [Sus
scrofa]
(SEQ ID NO: 10)
MWSLVMRLALSLAGTGAAPPIQSRIIGGRECEKDSHPWQVAIYHYSSFQC
GGVLVDPKWVLTAAHCKNDNYQVWLGRHNLFENEVTAQFFGVTADFPHPG
FNLSLLKNHTKADGKDYSHDLMLLRLQSPAKITDAVKVLELPTQEPELGS
TCQASGWGSIEPGPDDFEFPDEIQCVELTLLQNTFCADAHPDKVTESMLC
AGYLPGGKDTCMGDSGGPLICNGMWQGITSWGHTPCGSANKPSIYTKLIF
YLDWINDTITENP

### Methods for Generating KLK1

Tissue kallikrein can be made synthetically, recombinantly or isolated from natural sources.

In embodiments, Porcine Tissue Kallikrein may be isolated from pig pancreas as described in the literature. Alternatively, porcine KLK1 may be purchased from various vendors such as Lee Bio Solutions (St. Louis, Mo., USA) Catalogue No: 314-10, CAS No: 9001-01-8, EC No: 3.4.21.35, or Chongqing Waycome Pharmaceutical, (Chongqing, Dianjiang, China). Human tissue kallikrein may be purchased from vendors such as Lee Bio Solutions (St. Louis, Mo., USA) Catalogue No: 314-15, CAS No: 9001-01-8, EC No: 3.4.21.35).

Alternatively, recombinant DNA technology may be used to generate recombinant KLK1. In other embodiments, DNA sequences encoding a human KLK1 polypeptide of the present invention were isolated and characterized. Further, human DNA may be utilized in eukaryotic and prokaryotic expression systems to provide isolatable quantities of KLK1 protein having biological and immunological properties of naturally-occurring KLK1 as well as in vivo and in vitro biological activities, in particular therapeutic activity, of naturally-occurring KLK1.

Prokaryotic or eukaryotic host expression (e.g., by bacterial, yeast and mammalian cells in culture) of exogenous DNA of the present invention obtained by genomic or cDNA cloning or by gene synthesis yields recombinant human KLK1 polypeptides described herein. KLK1 polypeptide products of cell culture expression in vertebrate (e.g., mammalian and avian) cells may be further characterized by freedom from association with human proteins or other contaminants, which may be associated with KLK1 in its natural mammalian cellular environment or in extracellular fluids such as plasma or urine. Products of typical yeast (e.g., Saccharomyces cerevisiae) or prokaryote (e.g., E. coli) host cells are free of association with any mammalian proteins. Depending upon the host employed, polypeptides of the invention may be glycosylated with mammalian or other eukaryotic carbohydrates or may be non-glycosylated. Polypeptides of the invention may also include an initial methionine amino acid residue (at position-1).

Illustrative of the present invention are cloned DNA sequences of human species origins and polypeptides suitably deduced therefrom which represent, respectively, the primary structural conformation of KLK1 of human species origins.

The cell culture expressed KLK1 polypeptides of the present invention may be isolated and purified by conventional means including, e.g., chromatographic separations or immunological separations involving monoclonal and/or polyclonal antibody preparations, or using inhibitors or substrates of serine proteases for affinity chromatography. Polypeptide products of the invention may be “labeled” by covalent association with a detectable marker substance (e.g, radiolabels, e.g., as I¹²⁵ or P³² and nonisotopic labels, e.g., biotin) to provide reagents useful in detection and quantification of KLK1 in solid tissue and fluid samples such as blood or urine. DNA products of the invention may also be labeled with detectable markers (for example, radiolabels such as I¹²⁵ or P³² and nonisotopic labels such as biotin) and employed in DNA hybridization processes to locate the KLK1 gene position and/or the position of any related gene family in a human, monkey and other mammalian species chromosomal map. The labeled DNA may also be used for identifying the KLK1 gene disorders at the DNA level and used as gene markers for identifying neighboring genes and their disorders.

Tissue kallikrein polypeptide products provided by the invention are products having a primary structural conformation of a naturally-occurring tissue kallikrein to allow possession of one or more of the biological properties thereof KLK1 is a serine protease which cleaves low-molecular-weight kininogen resulting in the release of kallidin (lys-bradykinin) This activity of KLK1 may be measured in an enzyme activity assay by measuring either the cleavage of low-molecular-weight kininogen, or the generation of lys-bradykinin. Assays include examples wherein a labeled substrate is reacted with KLK1, and the release of a labeled fragment may be detected. One example of such a fluorogenic substrate suitable for KLK1 measurement of activity is D-val-leu-arg-7 amido-4-trifluoromethylcoumarin (D-VLR-AFC, FW 597.6) (Sigma, Cat #V2888 or Ana Spec Inc Cat #24137.) When D-VLR-AFC is hydrolyzed, the free AFC produced in the reaction can be quantified by fluorometric detection (excitation 400 nm, emission 505 nm) or by spectrophotometric detection at 380 nm (extinction coefficient=12,600 at pH 7.2). Other methods and substrates may also be used to measure KLK1 proteolytic activity.

KLK1 activity, measured in Units or Units/ml, may be determined by comparing the relative activity of a KLK1 sample to the Kininogenase, Porcine standard acquired from the National Institute for Biological Standards and Control (NIBSC Product No. 78/543). For this standard, the assigned potency is 22.5 international units (IU) per 20 μg ampoule of porcine pancreatic kininogenase. Typically, serial dilutions are made of the standard, and the activity in an unknown sample of KLK1 is compared to the standard.

### Administration of Tissue Kallikrein-1

In one aspect, a method of treating a human subject with β cell dysfunction comprises administering about 0.01 to about 100 IU per day of tissue kallikrein-1 (KLK1), a variant of active fragment thereof to about 100 the subject. The subject can be identified for increased risk of diabetes based on family history, the presence of autoantibodies, or may have signs of impaired glucose metabolism.

In embodiments, Tissue kallikrein-1, a variant, or active fragment thereof can be administered to a subject to increase islet cell numbers in vivo. More specifically, tissue kallikrein-1, a variant, or active fragment thereof can be administered to a subject to increase β-cells in vivo. It is believed that growth of β-cells results in increased production of insulin. As exemplified herein, administration of tissue kallikrein-1, a variant, or active fragment thereof can also increase the tolerance of glucose.

In type II diabetes, either a subject is not making enough insulin or a subject has become resistant to insulin. Administration of tissue kallikrein-1, a variant, or active fragment thereof can treat this condition. The classic example of a subject not making enough insulin is in an obese individual, where the demand has outgrown the supply of insulin. Administration of a tissue kallikrein-1 polypeptide can stimulate proliferation and increase the number of islet cells, and specifically the β cells. With such an increase, more insulin can be produced to try to meet the demand of a subject.

In an embodiment, tissue kallikrein, a variant, or active fragment thereof can be administered to a subject newly diagnosed diabetes, especially in subjects with type I diabetes. Newly diagnosed subjects with type I diabetes may not have a complete destruction, functional inhibition, or depletion of β-cells. Administration of tissue kallikrein, a variant, or active fragment thereof can stimulate the remaining β-cells to proliferate and produce insulin. Early administration of tissue kallikrein, a variant, or active fragment thereof can be beneficial to maximize the remaining healthy β-cells.

A subject may be a mammal, preferably a human, and may be a clinical patient, a clinical trial volunteer, and the subject may be suspected of having or being at risk for having diabetes, or be diagnosed with diabetes, or be a control subject that is confirmed to not have diabetes. In one preferred embodiment, a subject is a mammal, most preferably, a human, suspected of having or being at risk for having diabetes, or be diagnosed with diabetes.

Route of administration. Tissue kallikrein-1 and active fragments thereof can be used in methods described herein including administering to a human patient in accord with methods known to medical practitioners, such as by intravenous administration, e.g., as a bolus or by continuous infusion over a period of time, by subcutaneous, intramuscular, intra-arterial, intraperitoneal, intrapulmonary, generally by intravenous or subcutaneous administration. In an embodiment, a kallikrein polypeptide can be administered by subcutaneous injection or intravenous infusion with 0.9% sodium chloride solution as an infusion vehicle.

Pharmaceutical Formulations. Methods well known in the art for making formulations are found in, for example, Remington: The Science and Practice of Pharmacy, Gennaro, ed., 21^{st} edition (2006). Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel. For therapeutic or prophylactic or preventative compositions, the compounds are administered to an individual in an amount sufficient to prevent or treat diabetes.

An embodiment includes a lyophilized formulation of tissue kallikrein or active fragment thereof adapted for subcutaneous administration as known in the art. Lyophilized formulations may be reconstituted with a suitable diluent to a high protein concentration and the reconstituted formulation may be administered subcutaneously to a mammal to be treated herein.

A formulation herein may also contain more than one active compound as necessary for the particular indication being treated, preferably those with complementary activities that do not adversely affect each other.

Active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions.

The formulations to be used for in vivo administration must be sterile. This can readily be accomplished by filtration through sterile filtration membranes.

Typically, pharmaceutical compositions comprises tissue kallikrein, a variant, or active fragments thereof and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. Pharmaceutical compositions comprising tissue kallikrein or active fragments thereof can be delivered as, for example, ageratum, sprays, oral suspensions, suppositories, eye drops, and injectable suspensions.

Appropriate doses will depend on the particular cell type, tissue, organ, or subject being treated. When administered to a subject, dose will depend on the particular mammal being treated (e.g., human or nonhuman primate or other mammal), age and general condition of the subject to be treated, the severity of the condition being treated, the particular therapeutic polypeptide, protein, or oligonucleotide in question, its mode of administration, among other factors. The initial dose may be larger, followed by smaller maintenance doses.

For any composition or formulation used in the methods of the invention, the therapeutically effective dose can be estimated initially from animal models. Dose-response curves derived from animal systems are then used to determine testing doses for the initial clinical studies in humans. In safety determinations for each composition, the dose and frequency of administration should meet or exceed those anticipated for use in the clinical studies. Toxicity and therapeutic efficacy of the compositions and formulations of the invention can be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it can be expressed as the ratio ED₅₀/LD₅₀. Compositions that exhibit large therapeutic indices are preferred.

The data obtained from the animal studies can be used in formulating a range of doses for use in humans. The therapeutically effective doses for human administration lay preferably within a range of circulating concentrations that include the ED₅₀ with little or no toxicity. The dosage can vary within this range depending upon the dosage form employed and the route of administration utilized.

In the studies listed herein, rats were administered a dose of KLK1 of 0.2 U, 1.0 U and 5.0 U. The rats had an approximate body weight of 0.300 to 0.400 kg. to calculate the dose administered to the rats on a per kilogram bases, the number of units of KLK1 was divided by the weight of the rat (0.2 U KLK1 /0.300 kg rat=0.67 U/kg; 0.2 U KLK1/0.400 kg rat=0.50 U/kg), and thus the dosages were:

0.2 U KLK1=0.50 to 0.67 U/kg;

1.0 U KLK1=2.5 to 3.3 U/kg; and

5.0 U KLK1=12.5 to 16.7 U/kg.

According to the FDA Guidance for Industry (Estimating the maximum safe starting dose in initial clinical trials for therapeutics in adult healthy volunteers, July 2005) the human equivalent dose (HED) is determined by multiplying the rat dose by 0.162 (ie 0.50 U/kg (rat)×0.162=0.08 U/kg (HUD)). As such, the human equivalent dose (HED) would be:

0.2 U KLK1 (rat)=0.08 to 0.11 U/kg (HED);

1.0 U KLK1 (rat)=0.41 to 0.53 U/kg (HED); and

5.0 U KLK1 (rat)=2.0 to 2.7 U/kg (HED).

As will be evident to those skilled in the art, the HED dose is an estimated human dose for healthy humans. As such, the therapeutic dose of KLK1 may be higher or lower.

A pharmaceutical composition formulated for subcutaneous, intramuscular or intravenous administration comprises about 0.01 to 100 IU of KLK1, or a variant or an active fragment thereof, optionally further comprising a pharmaceutically acceptable excipient. A dose of KLKI, variant, or active fragment thereof, can be a dose of about 0.01 to about 100 IU per kg per day; about 0.01 to about 75 IU per kg per day; about 0.01 to about 50 IU per kg per day; about 0.01 to about 40 IU per kg per day; about 0.01 to about 30 IU per kg per day; about 0.01 to about 25 IU per kg per day; about 0.01 to about 20 IU per kg per day; about 0.01 to about 15 IU per kg per day; about 0.01 to about 10 IU per kg per day; about 0.01 to about 7.5 IU per kg per day; about 0.01 to about 5.0 IU per kg per day; about 0.01 to about 2.5 IU per kg per day; about 0.01 to about 2.0 IU per kg per day; about 0.01 to about 1.5 IU per kg per day; about 0.01 to about 1.0 IU per kg per day; about 0.01 to about 0.5 IU per kg per day; about 0.05 to about 10 IU per kg per day; about 0.10 to about 10 IU per kg per day; about 1.5 to about 10 IU per kg per day; about 2.0 to about 10 IU per kg per day; about 2.5 to about 10 IU per kg per day; about 5.0 to about 10 IU per kg per day; about 7.5 to about 10 IU per kg per day; about 0.1 to about 100 IU per kg per day; about 0.15 to about 10 IU per kg per day; about 0.20 to about 10 IU per kg per day; about 0.25 to about 10 IU per kg per day; about 0.30 to about 10 IU per kg per day; about 0.40 to about 10 IU per kg per day; about 0.50 to about 10 IU per kg per day; about 0.75 to about 10 IU per kg per day; about 0.10 to about 10 IU per kg per day; about 1.0 to about 25 IU per kg per day; about 1.0 to about 50 IU per kg per day; about 5.0 to about 25 IU per kg per day; about 5.0 to about 50 IU per kg per day; about 10 to about 25 IU per kg per day; about 10 to about 50 IU per kg per day; or about 25 to about 75 IU per kg per day. Treatment may involve administration of a single dose or multiple doses. In different embodiments, the dosage may be calculated on the amount or weight of KLK1 present. To determine a dose based on the amount or weight of KLK1 present, the specific activity of the KLK1 preparation would need to be calculated by comparing the specific preparation of KLK1 to the standard, which is assigned an activity of 22.5 international units (IU) per 20 μg, as described above. As will be evident to those skilled in the art, the dosage based on the amount of KLK1 may vary if the dosage is based on the amount of active (IU's) KLK1 that is administered since there may be differences in activities between different preparations of KLK1.

The above disclosure generally describes the present disclosure, which is further described by the following examples. These specific examples are provided solely for purposes of illustration, and are not intended to limit the scope of this disclosure. Although specific targets, terms, and values have been employed herein, such targets, terms, and values will likewise be understood as exemplary and non-limiting to the scope of this disclosure.

### EXAMPLES

### Example 1

### The Effect of Administration of Tissue Kallikrein-1 (KLK1) on β Cells in the Pancreas of a Rat Model of Diabetes

### Methods

Experimental diabetes was induced in male Wistar rats (8 weeks of age) by intraperitoneal (i.p.) injection of 50 mg/kg of streptozotocin (STZ) (in citrate buffer pH 4.5; 0.05M). Induction was designated as Day 1 (t=0-24 hours; STZ administered at t=0). For control rats, phosphate buffered saline (PBS) was injected in place of STZ. Rats were allocated into the following 9 treatment groups (n=8/group) on Day 5: 1) no STZ, PBS BID intramuscular (i.m.), 2) STZ+PBS BID i.m., 3) STZ+0.2 U KLK1 BID i.m., 4) STZ, 1.0 U KLK1 BID i.m., and 5) STZ, 5.0 U KLK1 BID i.m. The various treatments were carried out daily from Day 7 to Day 28. Rats were further subjected to daily 5-bromo-2-deoxyuridine (BrdU) injections (50 mg/kg; i.p.) from Day 7 to Day 15 to label cells undergoing S-phase for assessment of β-cell proliferation. Rats were fed a diet of standard Purina chow during the course of the experiments.

Blood analysis. For blood sample analysis, fasting blood glucose was evaluated from blood samples taken on Days 5, 8, and 14, (after overnight fasting) and 21, 27 (after 6 hour fasting). Oral glucose tolerance tests (0-120 min) were carried out on Days 14, 21, and 27. Blood (150 μl) was collected for each time point in K2EDTA-containing tubes. Insulin assays were carried out by ELISA on Day 27 OGTT blood samples.

Organ Harvesting. Rats were sacrificed at the end of the treatment period (Day 28) Pancreata were harvested and fixed (head portion) or processed for downstream measurements (tail portion). Rather than cutting longitudinally, pancreata were separated between the tail and head sections. The tail section was used to determine insulin content (μg/pancreas) and the head portion fixed for analysis of beta cell area/mass, islet number, beta cell proliferation, and insulin-positive duct cells. Pancreata were fixed in formalin and small intestine samples were embedded in paraffin and sectioned to obtain 5 μm sections. One serial section was stained with hematoxylin and eosin and another serial section underwent immunohistochemistry.

Pancreatic/β-cell area: Two typical 4× objective fields for each IHC sample were photographed at a fixed pixel density that contained all or most of the pancreas head region with representative areas of islets. Nikon Elements 3.0 software was used to threshold these images for insulin-positive tissue (red tissue) and the number of insulin-positive pixels was recorded. The image was then thresholded for all pancreatic tissue present (excluding white space and all non-pancreatic tissue such as small intestine or lymph node) and the number of pixels recorded. The area of insulin-positive tissue was reported as: area of insulin-positive tissue/area of all pancreatic tissue in the image. Data are presented as the sum of two data points.

Pancreatic/β-cell mass index: The pancreatic β-cell mass index was determined as the percentage of insulin-positive tissue reported for the pancreatic β-cell area above multiplied by the total weight of the pancreas to arrive at a pancreatic β-cell mass index.

Islet numbers: Islet number was determined using the same two 4× objective fields for each sample used to assess pancreatic β-cell area. The number of islets was counted and recorded.

Pancreatic/β-cell proliferation: An anti-BrdU antibody was conjugated to DAB as a chromagen (brown) and an anti-insulin antibody conjugated to fast red as a chromagen (red). Brown BrdU-positive nuclei within insulin-positive cells of islets were quantified. The number of these cells in 5 evenly-sized large islets was counted and recorded.

Number of insulin positive duct cells: To determine the number of insulin-positive duct cells, five 20× objective fields with large pancreatic ducts were examined. Any insulin-positive duct cells in these 5 fields were counted and recorded.

### Results

The effect of KLK1 on β-cell area, β-cell mass index, islet numbers, and proliferation was assessed in male Wistar rats corresponding to the groups: 1) no STZ+PBS, 2) STZ+PBS, 3) STZ+0.2U KLK1, 4) STZ+1.0U KLK1, and 5) STZ+5.0U KLK1.

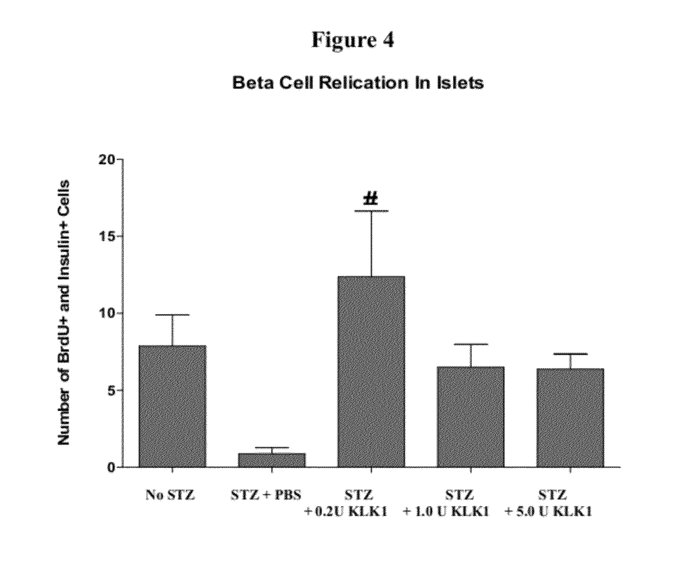

β cell replication in Islets. STZ treatment resulted in a decrease in number of BrdU⁺ insulin⁺ cells (i.e., proliferating β-cells) compared to no STZ group, indicating that STZ inhibited β-cell replication in islets (FIG. 4). The number of BrdU⁺ insulin⁺ cells was significantly higher in the STZ+0.2 U KLK1 group compared to the STZ+PBS group (1277% increase; p<0.01, ANOVA with Tukey's post hoc test), indicating a significant increase in β-cell proliferation (FIG. 4). The other KLK1 dosage groups also exhibited increased β-cell proliferation compared to the STZ+PBS group. Evidence of an increase in replicating β-cells could also be seen in micrographs (FIG. 21).

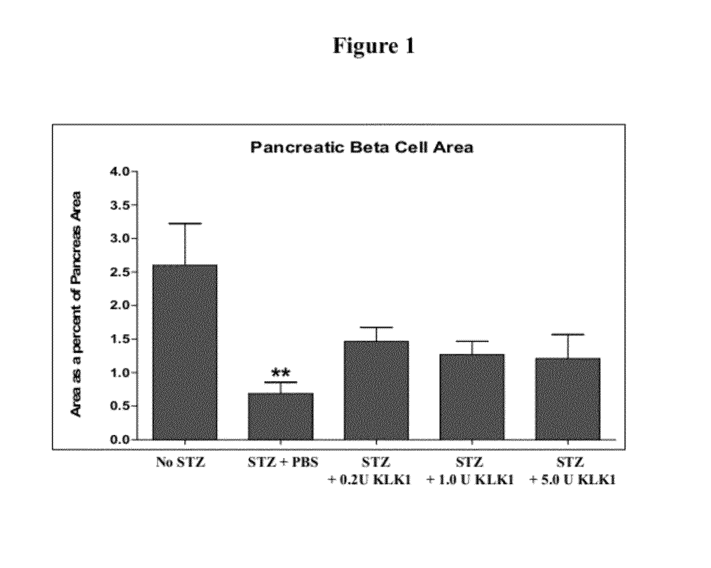

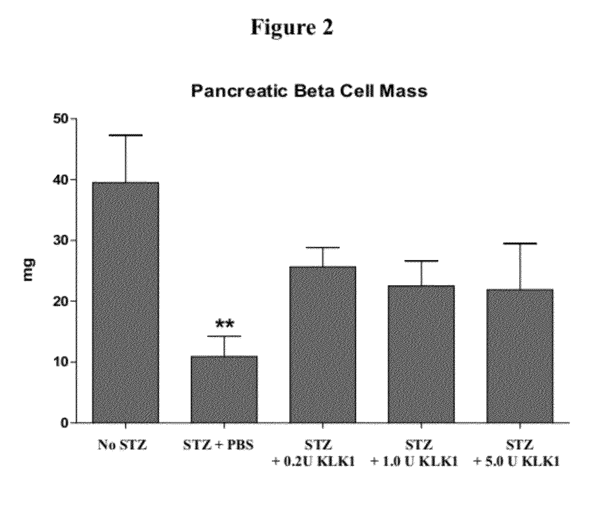

β cell area, β cell mass, Islet Cell number, and Insulin Positive Duct Cells. Pancreatic β cell area, β cell mass, and islet cell number were significantly reduced in the STZ+PBS group compared to the control no STZ group, indicating that STZ successfully induced islet atrophy (p<0.01) (FIG. 1-3). All three dosage groups of KLK1 in STZ treated rats showed increased β-cell area and β cell mass compared to the STZ+PBS group (FIGS. 1 and 2). However, the trend shows an increase in β cell area and mass in the KLK1 treated rats.

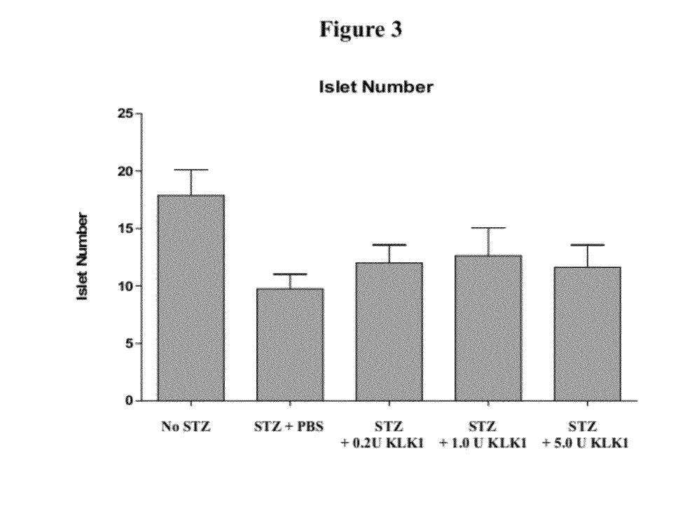

The results for Islet Cell number in the KLK1 treated rats were not noticeably different from STZ treated rats (FIG. 3). With increases in β cell number and β cell area, the tissue kallikrein effect appears to be specific for β cells.

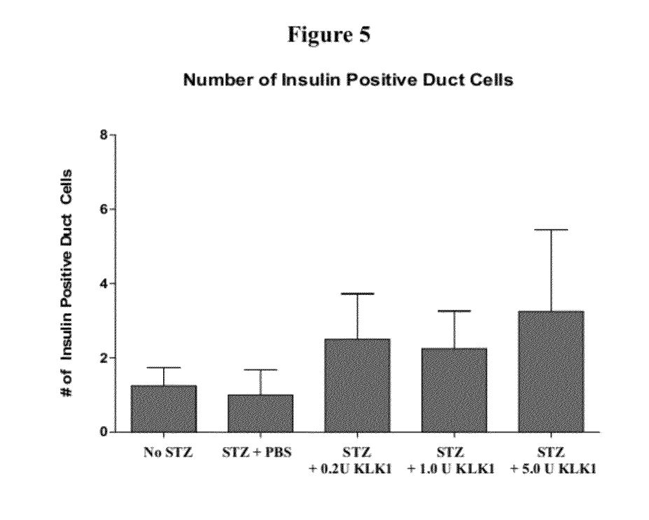

The number of insulin-positive duct cells was slightly reduced in the STZ+PBS group compared to the no STZ group (FIG. 5). Although the number of insulin positive duct cells in the STZ treated rats was only slightly decreased form the untreated rats, the KLK1 treated rats showed an increase in the number of duct cells in the three KLK1 dosage groups compared to the STZ+PBS group (FIG. 5).

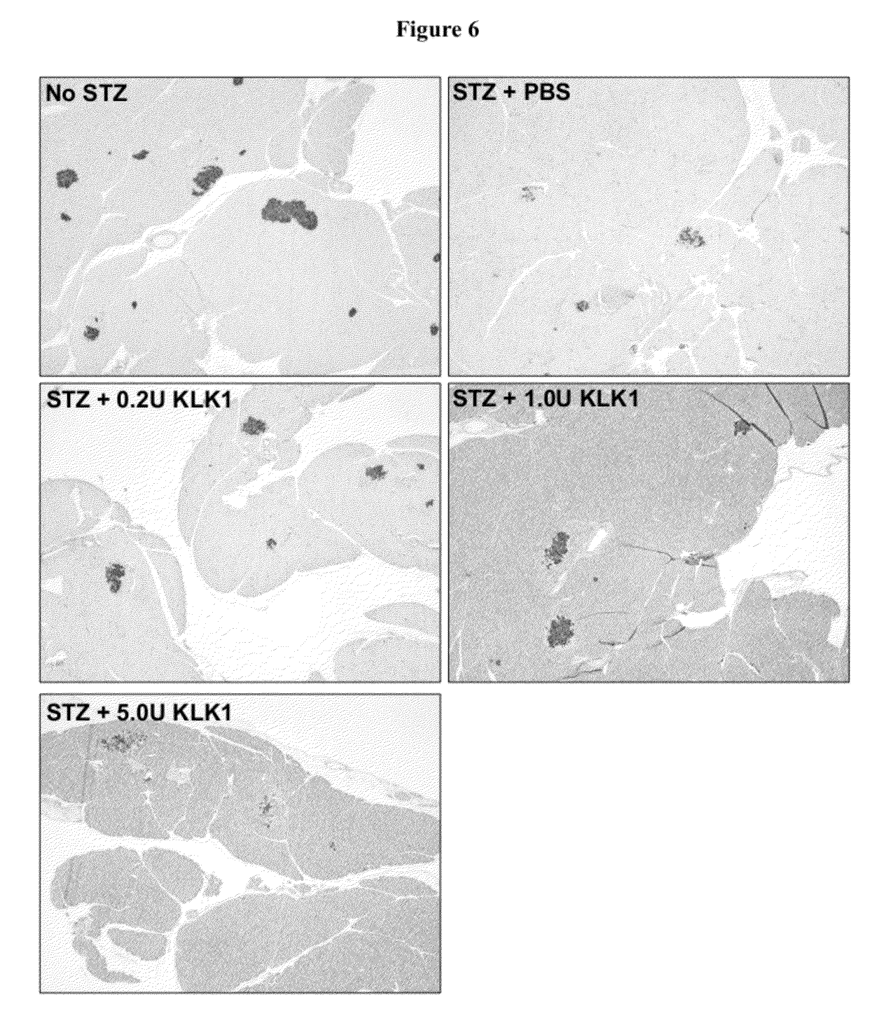

The above findings were supported by histopathological analysis (see FIG. 6). Rats in the no STZ group had normal pancreata, with only rare pancreatic duct cells positive for insulin via immunohistochemistry. Atrophy of islets is evident in the STZ+PBS group. Islets in the STZ+PBS group were smaller and less frequent in number than those in the no STZ group. Furthermore, many of the islets positive for insulin in the STZ+PBS group had reduced area compared to the no STZ group, with rare pancreatic duct cells positive for insulin. With respect to the various KLK1 dosage groups, islets were still atrophied, but less so compared to the STZ+PBS group, supporting the finding that KLK1 increases insulin positive β-cell proliferation. Only rare pancreatic duct cells were positive for insulin. STZ treated rats are a recognized animal model for diabetes. The effect of KLK1 on β cell proliferation in the STZ treated rats was studied. Changes in β-cell area, β-cell mass, islet numbers, and number of BrdU⁺ insulin⁺ cells was determined. However, a statistically significant increase in β cell proliferation was observed at 0.2 U dose of KLK1 in STZ treated rats. This increase in β cell proliferation is supported by the data that show an increase in β cell area and mass as well as the number of insulin positive duct cells in KLK-1 treated rats.

### Example 2

### KLK1-induced Decrease in Fasting Blood Glucose and Terminal Blood glucose in STZ-treated Rats

Fasting blood glucose levels and terminal blood glucose levels were compared in STZ rats treated with KLK1. If fasting blood glucose is high that is an indication that insulin levels are low or impaired.

### Methods

Fasting blood glucose measurements: The effect of KLK1 on fasting blood glucose levels was assessed in male Wistar rats corresponding to the following groups (n=8/group): 1) no STZ, PBS BID intramuscular (i.m.), 2) STZ(50 mg/kg)+PBS BID i.m., 3) STZ+0.2 U KLK1 BID i.m., 4) STZ, 1.0 U KLK1 BID i.m., and 5) STZ, 5.0 U KLK1 BID i.m. . Fasting glucose experiments were conducted on Days 5, 8, 14, 21, and 27, with fasting glucose on Day 5 being used to assign the rats in the dosing groups. Rats were fasted for 12 hours on Day 5, 8, and 14 and 6 hours on Days 21 and 27.

Rats were allocated into the various dosage groups such that the mean fasted blood glucose per group was approximately 105 mg/dL on a random basis on Day 5. Blood samples were obtained by tail nicking of conscious rats. On the day of the test, rats were placed into their appropriate spots in the restrainers, and tails were clipped. Thirty minutes later, rats were treated as indicated above. Thirty minutes post-administration, tails were clipped and blood was collected. Fasting glucose readings were taken with a glucose meter and strip system (Bayer Ascensia Contour meter and glucose strips).

### Results

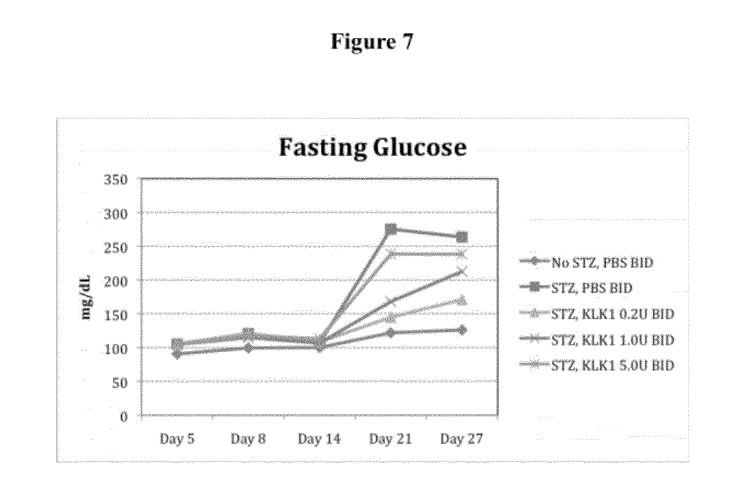

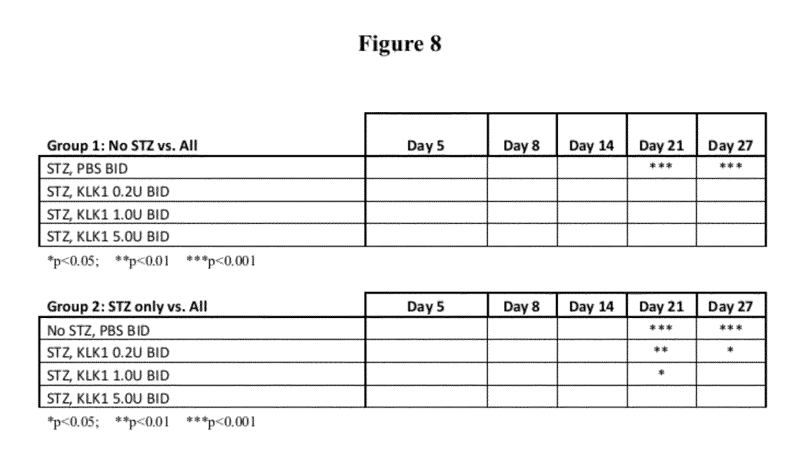

Fasting blood glucose significantly increased in the STZ+PBS group compared to the no STZ group on Days 21 and 27 (FIG. 7). Fasting blood glucose was significantly lower in the 0.2 U KLK1, 1.0 U KLK1, and 5.0 U KLK1 groups as compared to the STZ treated group.

These results demonstrate that KLK1 is effective in lowering glucose levels in a STZ rat model.

### Example 3

### KLK1-induced Increase in Pancreas Insulin Levels in STZ-treated Rats

### Methods

The effects of KLK1 on pancreas insulin levels were assessed in male Wistar rats corresponding to the following groups (n=8/group): 1) no STZ, PBS BID intramuscular (i.m.), 2) STZ+PBS BID i.m., 3) STZ+0.2 U KLK1 BID i.m., 4) STZ, 1.0 U KLK1 BID i.m., and 5) STZ, 5.0 U KLK1 BID i.m.

Insulin levels (μg/pancreas) were determined from pancreas tails harvested on Day 28 in the dosing groups 1) no STZ+PBS, 2) STZ+PBS, 3) STZ+0.2 U KLK1, 4) STZ+1.0 U KLK1, 5) STZ+5.0 U KLK . Insulin was extracted by acidified ethanol extraction and levels measured using an ELISA kit (Crystal Chem, 90060, Ultra Sensitive Rat Insulin Kit) for rat insulin.

### Results

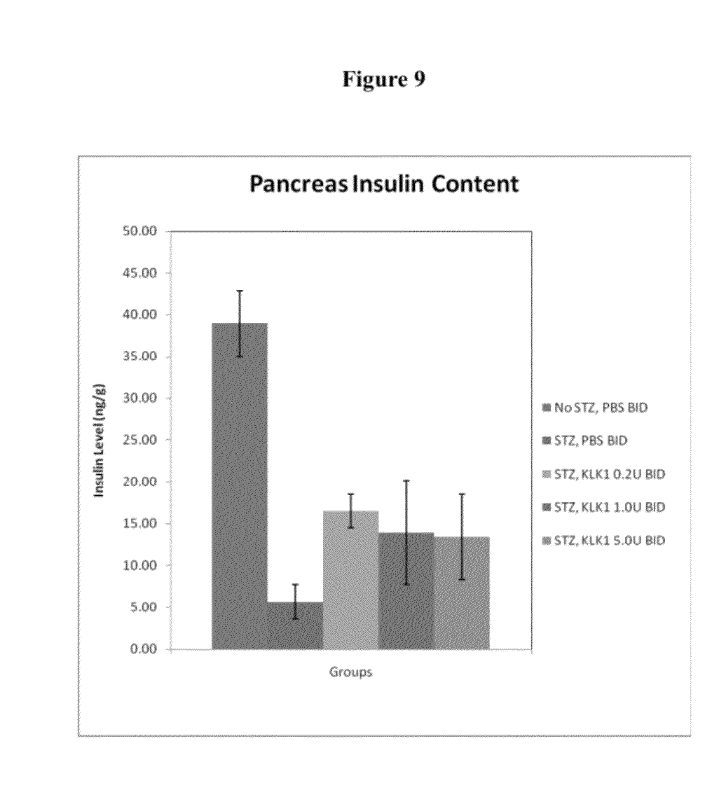

Pancreatic insulin levels decreased in the STZ+PBS group compared to the no STZ group, reflecting the loss of β-cells which produce insulin (p<0.001, FIG. 9). Pancreas insulin levels increased in the various KLK1 dosage groups compared to the STZ+PBS group.

These results indicate that KLK1 treatment produce an increase in insulin levels in the pancreas. One explanation for this increase in pancreatic insulin levels is that it correlates with an increase in the number of β cells which is also seen at 28 days after treatment.

### Example 4

### KLK1 Improves Glucose Tolerance in STZ-treated Rats

Oral glucose tolerance test provides a measure of glucose homeostasis. If the oral glucose tolerance test is high that is an indication that insulin levels are low or beta cells are impaired. Oral glucose tolerance levels were compared in STZ rats treated with KLK1 .

### Methods

Male Wistar rats corresponding to the following groups (n=8/group) were subjected to the oral glucose tolerance test (OGTT) to determine the effects of KLK1 on glucose levels in response to a glucose load: 1) no STZ, PBS BID intramuscular (i.m.), 2) STZ+PBS BID i.m., 3) STZ+0.2 U KLK1 BID i.m., 4) STZ, 1.0 U KLK1 BID i.m., and 5) STZ, 5.0 U KLK1 BID i.m.

Fasting blood glucose: An OGTT (0, 15, 30, 60, 90, and 120 min) was performed on Days 14, 21, and 27 for the following groups: 1) no STZ+PBS, 2) STZ+PBS, 3) STZ+0.2 U KLK1, 4) STZ+1.0 U KLK1, and 5) STZ+5.0 U KLK1. Glucose (1 g/kg at a dose of 5 ml/kg) was administered to rats by oral gavage. Rats were fasted for 12 hours on Day 14 and 6 hours on Days 21 and 27 prior to initiating the OGTT.

AUC calculations: AUCs were calculated using GraphPad PRISM version 4. PRISM computes AUC using the trapezoid rule and defines a curve as a series of connected X,Y points, with equally spaced X values. Baseline was defined as the mean of the initial values. Any negative area under the curve was not taken into account in reference to the calculations. AUC data were calculated for 0, 15, 30, 90, and 120 minute time points for each day OGTT was conducted. Both total AUC and positive AUC were calculated.

### Results

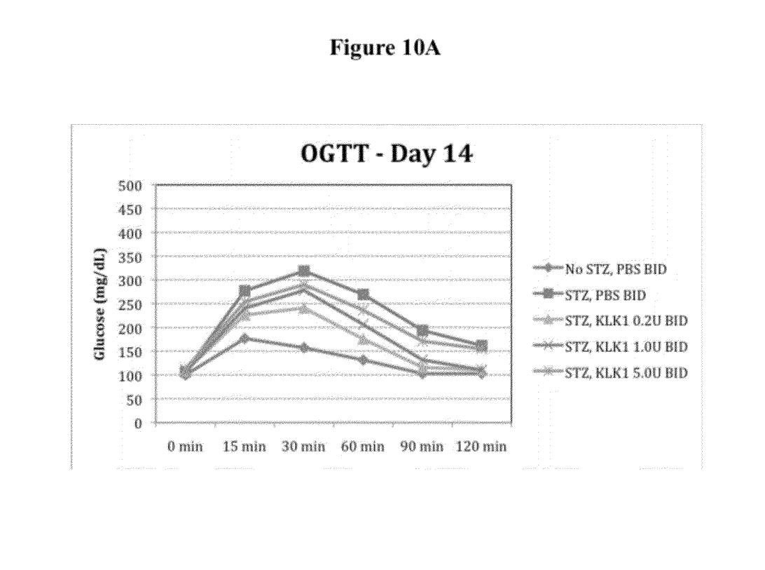

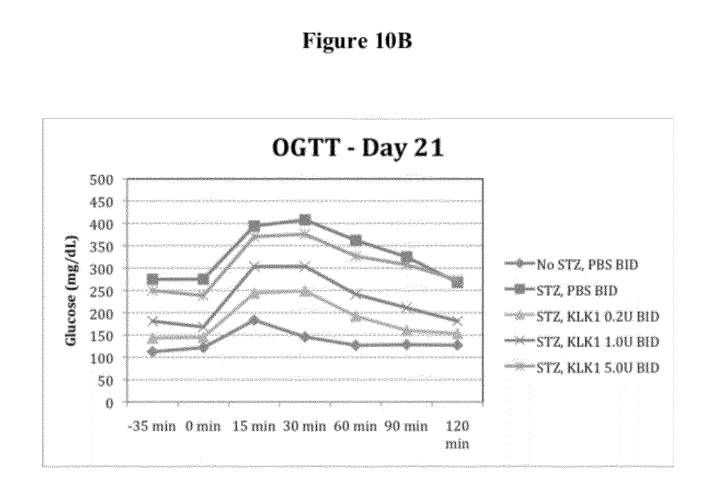

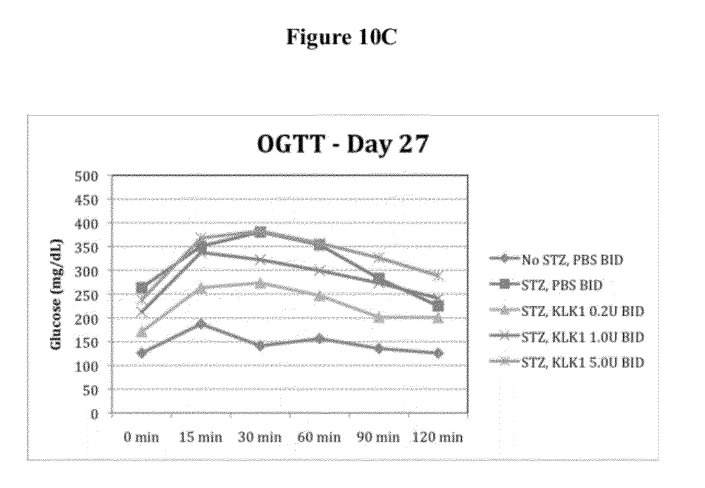

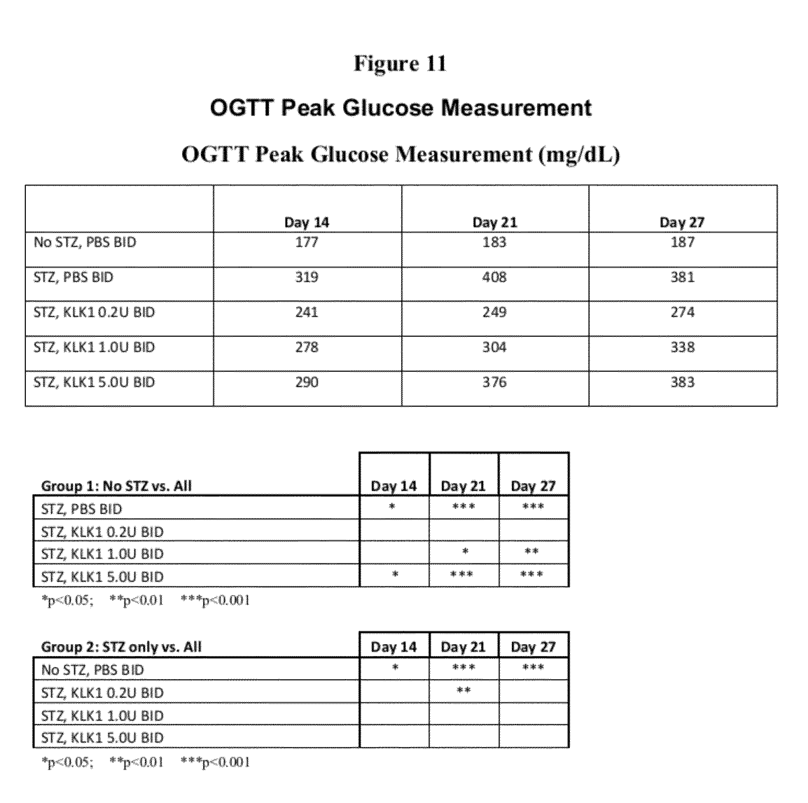

Rats in all KLK1 dosage groups tolerated glucose load better than the STZ+PBS group on Days 14 and 21 (FIG. 10A and 10B). At Day 27, rats in the 0.2 U KLK1, and 1.0 U KLK1 groups showed better tolerance to a glucose load compared to the STZ+PBS group (FIG. 10C). Consistent with this, peak glucose levels in response to the glucose load were lower in the 0.2 U KLK1 dosage group compared to the STZ group (FIG. 11).

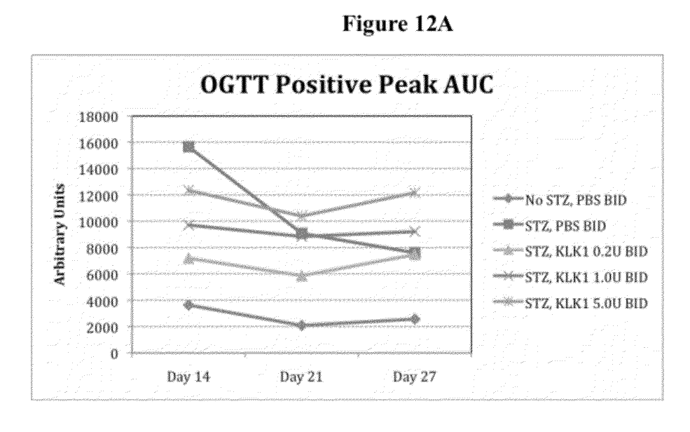

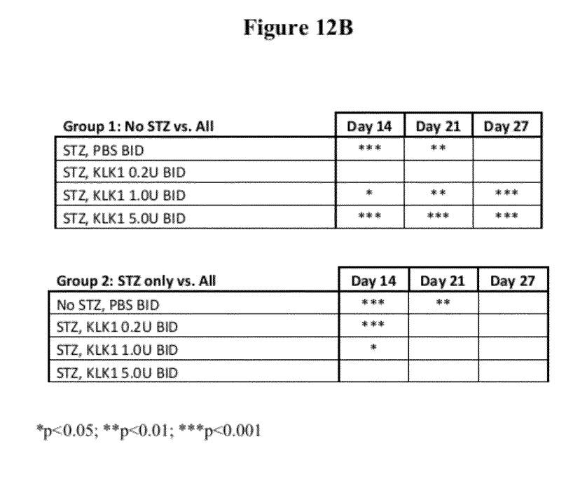

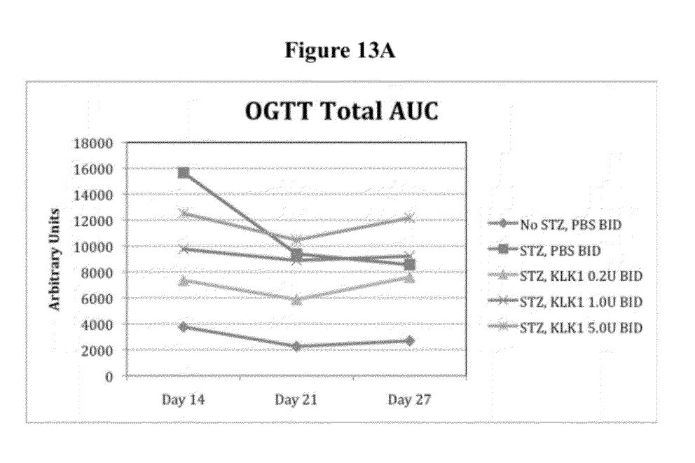

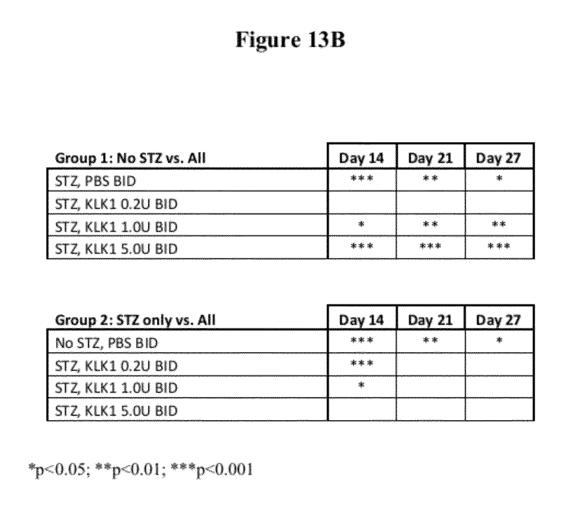

Positive peak area under the curve (AUC) was significantly lower for STZ rats treated with 0.2 U and 1.0 U KLK1 than untreated STZ rats (STZ+PBS) (FIG. 12). The KLK-1 dosage groups, except for the 0.2 U KLK-1 groups, showed statistically significantly higher positive peak AUC compared to the no STZ group on Days 14, 21, and 27 (FIG. 12, middle panel). Similar results were seen for the total area under the curve (AUC) (FIG. 13).

These graphs showed that KLK1 treated STZ rats were able to tolerate glucose better than non-KLK1 treated STZ rats. The results indicate that KLK1 can improve glucose tolerance. Potentially, KLK1 can be administered to improve glucose tolerance not only in individuals with diabetes but also individuals with prediabetes.

### Example 5

### KLK1-induced Increase in Postprandial Insulin Levels in STZ-treated Rats

The effects of KLK1 on postprandial insulin levels were assessed in male Wistar rats corresponding to the following groups: 1) no STZ, PBS BID intramuscular (i.m.), 2) STZ+PBS BID i.m., 3) STZ+0.2 U KLK1 BID i.m., 4) STZ+1.0 U KLK1 BID i.m., and 5) STZ+5.0 U KLK1 BID i.m.

### Methods

Blood insulin levels were assessed by ELISA (Crystal Chem, 90060, Ultra Sensitive Rat Insulin kit) using blood samples obtained from OGTT (0, 15, 30, 60, 90, and 120 min. blood samples) on Day 27 in the following groups: 1) no STZ+PBS, 2) STZ+PBS, 3) STZ+0.2 U KLK1, 4) STZ+1.0 U KLK1, and 5) STZ+5.0 U KLK1.

### Results

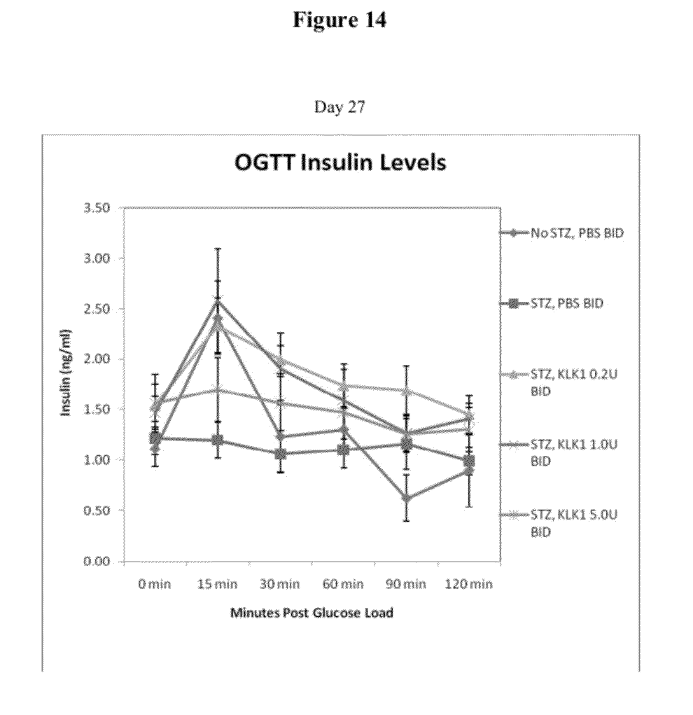

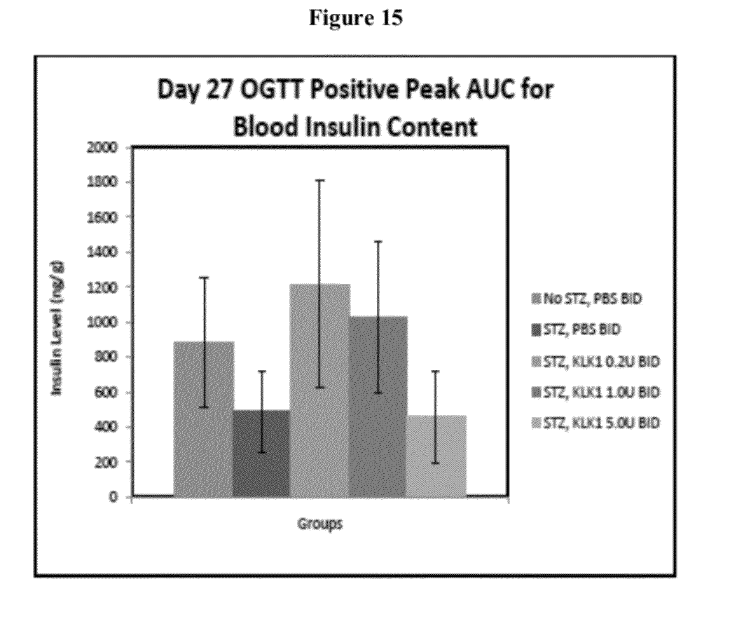

Blood insulin levels decreased in the STZ+PBS group compared to the no STZ group, (FIG. 14). Peak blood insulin levels (at 15 min) significantly increased and were in fact similar to the no STZ group in the STZ+0.2 U KLK1 group compared to the STZ+PBS group (p<0.05, ANOVA with Bonferroni post hoc test). Blood insulin also increased in STZ+1.0 U KLK1 and STZ+5.0 U KLK1 groups compared to the STZ+PBS group, although to a lesser extent than that observed in the STZ+0.2 U KLK1 group. In addition to the restoration of peak insulin levels, the KLK1 dosage groups showed higher post-prandial insulin levels at later time points (30, 60, 90, and 120 minutes) compared to the STZ+PBS group. Consistent with these results, Day 27 positive peak AUCs for blood insulin content were higher in the KLK1 dosage groups (i.e., 0.2 U and 1.0 U KLK1 groups) compared to the STZ+PBS group (FIG. 15). The data from testing postprandial insulin levels was consistent with KLK1 treatment effects in previous examples.

### Example 6

### Effects of KLK1 on Body Weight, Urinary Excretion of Glucose, Creatinine, Protein, and Albumin, and Kidney Hypertrophy

Male Wistar rats corresponding to the following groups were subjected to analysis of urinary excretion of glucose, creatinine, protein, and albumin, body weight, and kidney hypertrophy. The kidney is an organ that is profoundly affected by the progression of diabetes. Urine should not contain glucose since the kidneys should recycle all of the filtered glucose back into the bloodstream. Glycosuria is almost always caused by elevated blood glucose levels. The presence of creatinine in the blood suggests impaired glomerular filtration. Creatinine is a waste product that should be filtered from the blood and then appear in the urine. Urine albumin is measured by comparing the amount of albumin to the amount of creatinine in a single urine sample. Proteinuria, and specifically, increased albumin in the urine, is a sign of kidney damage.

### Methods

Body weight of rats in each group (n=8/group) was measured on Days 1, 7, 13, 20, and 28. Urinary glucose, creatinine, protein, and albumin excretion were measured on Day 25. Total kidney weight/body weight to assess kidney hypertrophy was measured (Day 28). These parameters were measured for the following groups: 1) no STZ, PBS BID intramuscular (i.m.), 2) STZ+PBS BID i.m., 3) STZ+0.2 U KLK1 BID i.m., 4) STZ, 1.0 U KLK1 BID i.m., and 5) STZ, 5.0 U KLK1 BID i.m.

### Results

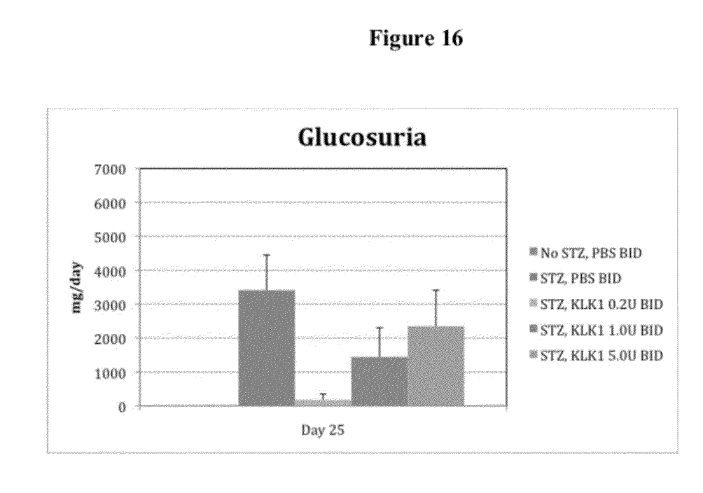

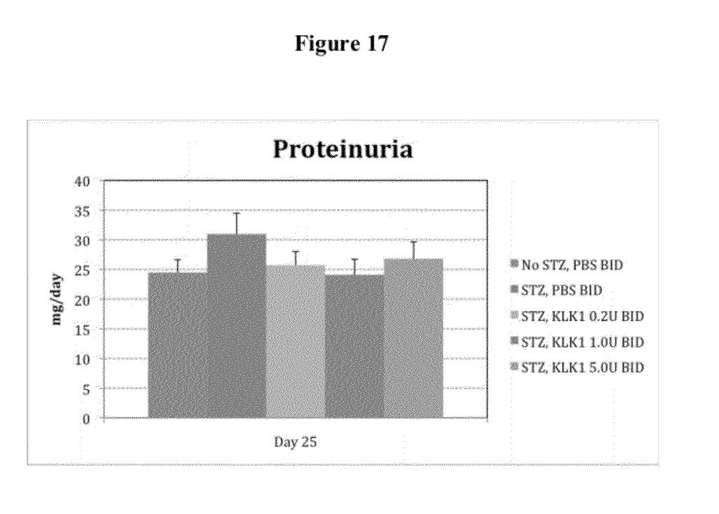

As expected, the negative control rats (no STZ) produced only trace amounts of glucose in their urine (FIG. 16). High glucose levels can be seen in the STZ treated rats. KLK1 treatment in the STZ treated rats reduced glucose excretion. In fact, 0.2 U KLK1 reduced glucose excretion by almost 95%.

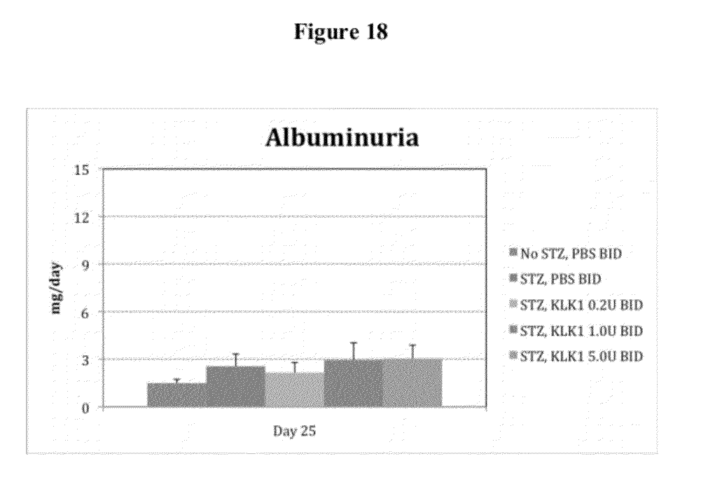

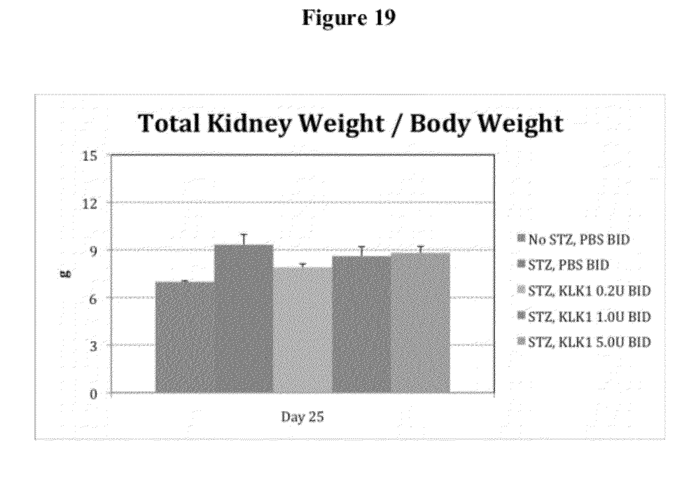

With regard to protein in the rats' urine, STZ treated rats excreted 126% more protein compared to the negative control of untreated rats (FIG. 18). All 3 concentrations of KLK1 treatments reduced protein excretion in the urine. Establishing the values for the negative control as a baseline, 0.2 U, 1.0 U, and 5.0 U of KLK1 treatment reduced protein excretion by 81%, 105%, and 64%, respectively. In regards to albumin in the rats' urine, STZ treated rats excreted 170% more albumin compared to the negative control of untreated rats (FIG. 19). The only treatments that reduced the level of albumin excretion were the 0.2 U KLK1.

Kidney hypertrophy is a clinical sign of kidney dysfunction that is prevalent in diabetes. Kidney hypertrophy was measured as total kidney weight as a percentage of body weight. STZ treatment of rats indicated kidney hypertrophy compared to the negative control of untreated rats (FIG. 20). All concentrations of KLK1 reduced the kidney hypertrophy that is caused by STZ treatment.

A determination of these nephrologic endpoints can provide additional information regarding the effectiveness of treating diabetes in the STZ rat diabetes model. Since earlier described results showed that KLK1 was successful in stimulating the proliferation of β cells with the concomitant increase in insulin secretion and lowered fasting blood glucose, the KLK1 treated rats also had better functioning kidneys as evidenced by improved glucosuria and proteinuria. KLK1 treatment results in better systemic outcomes for complications of diabetes.

While the disclosure has been described in each of its various embodiments, it is expected that certain modifications thereto may be undertaken and effected by the person skilled in the art without departing from the true spirit and scope of the disclosure, as set forth in the previous description and as further embodied in the following claims. The present disclosure is not to be limited in scope by the specific embodiments described herein. Indeed, various modifications of the disclosure in addition to those described herein will become apparent to those skilled in the art from the foregoing description and the accompanying figures. Such modifications are intended to fall within the scope of the appended claims. It is further to be understood that all values are approximate, and are provided for description.

1. A method of treating a human subject with β cell dysfunction comprising administering about 0.01 to about 100 IU per day of tissue kallikrein-1 (KLK1), a variant of active fragment thereof to about 100 the subject.

2. The method of claim 1 wherein the KLK1 is administered via subcutaneous, intramuscular, or intravenous administration.

3. The method of claim 1 wherein the subject is newly diagnosed with diabetes.

4. The method of claim 3 wherein the subject is treated to improve blood glucose levels.

5. The method of claim 3 wherein the subject is treated to stimulate proliferation of β cells.

6. The method of claim 1 wherein the amount of KLK1, a variant of active fragment thereof, is about 0.01 to about 10 IU per day.

7. A method of stimulating β cell proliferation comprising contacting β cells with an effective amount of tissue kallikrein-1 (KLK1), a variant or active fragment thereof

8. A method of stimulating β cell proliferation in a subject comprising administering an effective amount of tissue kallikrein-1 (KLK1), a variant or active fragment thereof, wherein the subject is newly diagnosed with diabetes.
